(19) United States
(12) Patent Application Publication
(10) Pub. No.: US 2013/0012955 A1
(43) Pub. Date: Jan. 10, 2013

(54) System and Method for Pedicle Screw Placement in Vertebral Alignment

(76) Inventor: Dean Lin, Fort Myers, FL (US)

(21) Appl. No.: 13/634,976

(22) PCT Filed: Feb. 25, 2011
(86) PCT No.: PCT/US11/26166

§ 371 Date: Sep. 14, 2012

Related U.S. Application Data
(60) Provisional application No. 61/314,619, filed on Mar. 17, 2010.

(51) Int. Cl. A61B 17/56

(52) U.S. Cl. 606/104

(57) ABSTRACT

A system and minimally invasive method for the placement of pedicle screws without the use of a trocar needle and/or guidewires. The system and method comprises at least one pedicle finder and at least one dilator. The pedicle finder preferably comprises an extender that is removably attached to a pedicle anchor. Attached to a pedicle anchor is a flexible tether. The extender preferably includes a passage through which the flexible tether extends. The dilator comprises a tubular body with two ends, where a first end comprises a means for securing the dilator into bony process of vertebra and a second end comprises at least two arm attachments. The tubular body Of the dilator is configured to slide easily over an extender and secure the first end into bony vertebra to prevent the dilator from dislodging. The arm attachment of the second end of the dilator can be interconnected with another arm attachment via a linking element and/or the dilator can be secured to a stable object such as a table via a securing element.

### CROSS-REFERENCE TO A RELATED APPLICATION

This application claims the benefit of U.S. provisional application Ser. No. 61/314,619, filed Mar. 17, 2010, which is incorporated herein by reference in its entirety.

### BACKGROUND OF THE INVENTION

In the past, surgical procedures, spinal surgical procedures in particular, were quite invasive, traumatic, and time consuming. Such surgeries typically utilized large incisions and extensive tissue retraction, where muscle and ligament tissues were retracted or surgically detached during surgery and reattached afterward. As a result, such surgeries lead to long recovery time, patient discomfort, an increased risk of infection, and high expense.

In an attempt to address these issues, minimally invasive surgical procedures have been devised. The advantages for the patient, when implementing such procedures, have been well documented with less pain, blood loss and tissue damage all contributing to a faster recovery and improved function with fewer complications. However, the smaller exposure of the surgical field has presented a challenge to the surgeon to accomplish the same goals of a successful open procedure with a technique having less direct visualization of the operative site.

All current minimally invasive techniques for placement of pedicle screws in vertebral alignment procedures require the use of guidewires, typically Kirschner or K wires. Such guidewires normally have a sharp tip and are driven into the bone, where access to the bone is provided by a small incision performed with a minimal open technique or a percutaneous technique. Intraoperative fluoroscopy, which provides real-time moving images of patient internal structures, is often used in conjunction to ensure safe placement of these wires. Cannulated instruments (e.g., awl, tap, etc.) then slide over these guidewires to introduce the pedicle screws into the pedicle and the vertebral body.

While necessary, the guidewires can serve as a source of great angst in minimally invasive vertebral alignment procedures. In such procedures, a trocar needle (e.g., jamshidi needle) is typically used to introduce a port for guidewire placement into bone. Unfortunately, trocar needles are often flimsy and prone to bending or breaking. In such circumstances, the clinician must expend additional time and effort to extract and replace the trocar, thereby increasing the likelihood of complications. Further, due to its flimsiness, the trocar needle can be difficult to properly dock in hard pedicle bone.

Once the guidewires are properly driven into bone, careful control of the wires must be maintained at all times or they can dislodge and come out of the pedicle, requiring re- placement of the wire and which necessitates additional fluoroscopy imaging. As understood by the skilled clinician, fluoroscopy involves the use of x-rays, a form of ionizing radiation, which poses a potential health risk to the patient and surgeon. Long length of exposure times to fluoroscopy can cause standard cancer-inducing stochastic radiation effects as well as deterministic radiation effects ranging from mild erythema to more serious burns.

Because guidewires pass through skin into bone, they form a potential passage for bacteria from the skin to migrate into the bone and cause infection. In certain instances, the sharp guidewires can be inadvertently pushed past the anterior vertebral body cortex and into the intra-abdominal cavity, with the potential to lacerate the great vessels or puncture the viscera. Guidewires can also back out of the bone losing the fixation and requiring reinsertion of guidewire and increasing the risk of infection and further complications. In addition, guidewires are easily bent, and any deformation of the wires may prevent the smooth placement of instruments over the wires and prevent screw placement completely.

Careful guidewire control must be maintained at all times and it is imperative that the surgeon knows exactly where the sharp tip of the guidewire is located all times. To do so, copious amounts of intraoperative fluoroscopy are often used during minimally invasive surgical procedures to ensure that guidewires are safely imbedded in bone, which exposes the patient, surgeon, the surgical assistant, and the entire OR staff to vast amounts of radiation.

Thus, the surgical practitioner today is faced with the choice between low exposure to radiation and a good view but increased tissue damage and patient complications, versus greater exposure to radiation with a much poorer view and heightened risk of improper insertion of the pedicle screw, but with potentially better patient outcome.

What is needed is a system that will enable easy, accurate and consistent placement of a pedicle screw without the need for a guidewire or trocar and which will not require extensive paraspinous muscle dissection for proper placement. The needed device will ideally reduce the risk of guidewire-associated complications as well as greatly decreasing patient, surgeon and staff exposure to fluoroscopic radiation.

### BRIEF SUMMARY OF THE INVENTION

The system and method of the invention illustrate a variety of structures and techniques to enable a staged location and entry into the pedicle for providing insertive, progressively larger threaded fixation and superior surgical control from a distance from the spine. The subject pedicle screw placement system can be used with any current imaging technology, which may include x-ray. or infrared, or radio frequency (RF) navigational guidance. This allows' for faster complex spine surgery procedures, decreased cost, decreased anesthesia time and complications such as surgery time dependent post operative infections. The systems and methods .of the invention can be utilized manually or power driven and can be used with open, minimal open, or percutaneous surgical procedures.

The subject pedicle screw placement system is simple and has a small number of components. These components include a pedicle finder and a dilator. The pedicle finder comprises: a pedicle anchor, a flexible tether attached to the pedicle anchor, and an extender removably attached to the pedicle anchor. The dilator is tubular in shape, having two ends and including a hollow passage through which an extender can easily traverse. The first end comprises means for securing the dilator into bone, such as several sharp protrusions or “teeth.” The second end comprises at least two attachment arms. In certain embodiments, linking elements can be used to lock the position of the dilators in relation to each other so as to use the pedicles themselves to anchor the dilators in place. A pedicle-based fixation system may obviate the need for other attachment arms by providing a solid, bony point of fixation. Because the mass and bulk of the soft tissue (i.e., muscle, fat, skin) can sometimes cause the dilator to move, thereby losing the exact position of the pedicle, the more points of fixation for the dilator, the more reliably the dilator can maintain its position over the pedicle. In addition, the pedicle-based fixation system can also serve as a means by which a minimally invasive tubular retractor can be. used. The retractor can provide visualization of the facet joint or lamina, by which a transforaminal approach to decompression can be performed (for a transforaminal lumbar interbody fusion (TLIF)).

The pedicle anchor comprises a sharp conical portion, which may be threaded along a portion or its entirety, to assist in advancing the pedicle anchor down into the bony pedicle, a means for attaching a flexible tether, and an extender attachment portion. The extender comprises a pedicle anchor attachment portion and a passage to accommodate the flexible tether. In certain embodiments, the extender includes a handle to enable rotational turning of the pedicle finder. Because of this configuration, a trocar is not needed to precisely dock a guidewire; nor is a guidewire needed to hold an entry point for a pedicle screw.

In a method of use, a pedicle finder is provided wherein a pedicle anchor is removably attached to an extender and the flexible tether traverses the extender passage to exit the extender. The sharp, threaded portion of the pedicle anchor is advanced into the patient to penetrate the cortical bone. Rotational turning of the extender transmits to the pedicle anchor, where the pedicle anchor is turned and screwed into bone. As the pedicle anchor is screwed into the. pedicle via rotational turning. of the extender, it gradually expands the cancellous inner bone (due to its threaded, conical shape) to not only securely embed the pedicle anchor in the. pedicle but also to tap a hole in the pedicle bone to facilitate later placement of a pedicle screw: In certain instances, further sequential tapping for driving a pedicle screw into bone is unnecessary following insertion and removal of a pedicle anchor of the invention.

Once the pedicle anchor is securely driven into the pedicle, the extender can be detached from the pedicle anchor and removed. The flexible tether, because it remains attached to the pedicle anchor, can be secured out of the way so that any decompression or interbody work that needs to be performed can proceed. In this manner, the flexible tether and pedicle anchor easily, safely, and securely maintain the location of the pedicle to which a pedicle screw is driven. In previous procedures, a guidewire or trocar needle would be used Unfortunately, either one requires maintenance of contact and manual tension by the clinician to ensure structural rigidity (to prevent bending or breakage), and thus, can be physiologically arduous and awkward for the surgeon to uphold while attempting to perform decompression or interbody work. In contrast, the flexible tether of the invention does not require such careful manipulation and tension; it merely requires securement away from the additional -- work to be performed.

Upon completion of any additional decompression or interbody work and/or when a pedicle screw is to be driven into a pedicle, an extender is reattached to the pedicle anchor merely by sliding the flexible tether through the extender passage and guiding the extender along the tether to the pedicle anchor. Once in contact with the pedicle anchor, the extender is easily reattached.

In certain instances, a dilator is then passed over the extender to prevent soft tissue from going into the threads of the tap following pedicle anchor removal and to enable a pedicle screw to be passed down the dilator and be screwed into the tap hole and bone.

According to the subject invention, at least one dilator can be interconnected to another via a linking element. A linking element comprises two ends and a lockable ball joint, where either end of the linking element easily attaches to a dilator arm. Alternatively, a dilator can be secured to a table or other stationary device using a fixation element.

Once all of the dilators are secured, the pedicle finder is detached from the pedicle by rotational turning of the extender, which transmits to the pedicle anchor and causes the pedicle anchor to unscrew from the bone. Once the pedicle anchor is free of the bone, the pedicle finder is removed from the dilator. While the dilator remains in place, in certain embodiments secured via linking element(s) and/or fixation element(s), a pedicle screw is slid down through the dilator passage and screwed into the tap hole of the pedicle provided by the pedicle anchor.

The pedicle screw placement system and method of the present invention provides significant advantages over the current and prior art. Namely, with the use of the subject technology, the skilled surgeon need not utilize a trocar needle and/or guidewire in placing a pedicle screw into bone. These and other features of the present invention will become more apparent from the following description of the embodiments and certain modifications thereof when taken with the accompanying figures.

### BRIEF DESCRIPTION OF THE DRAWINGS

FIG. 1 is of an embodiment of a pedicle finder of the invention.

FIG. 2 is of an embodiment of the invention of a pedicle anchor.

FIGS. 3A and 3B are embodiments of a pedicle finder of the invention in which an extender has been unfastened from a pedicle anchor and the extender is entirely separate from the pedicle anchor.

FIG. 4A and 4B are embodiments of a pedicle finder of the invention in which an extender has been unfastened from a pedicle anchor, where the tether is still retained within the extender.

FIG. 5 is of an embodiment of a dilator of the invention.

FIG. 6 is of an embodiment of several interconnected dilators of the invention.

FIG. 7 is of an embodiment of the invention in which a dilator is placed over an extender of a pedicle finder.

FIG. 8 is of an embodiment of the invention in which a dilator is secured over an extender of a pedicle finder.

FIG. 9 is of an embodiment of the invention in which several dilators secured over extenders of pedicle finders are interconnected.

FIG. 10 is of an embodiment of the invention in which a pedicle finder is removed from a dilator.

FIG. 11 is of an embodiment of the invention in which a pedicle screw is being placed into vertebra via stationed dilators.

FIG. 12 is an embodiment of the invention in which pedicle screws are appropriately situated.

FIGS. 13A and 13B are top and bottom views, respectively, of a dilator of the invention.

### DETAILED DESCRIPTION OF THE INVENTION

The subject invention provides improved systems and methods for pedicle screw placement. According to the subject invention, the systems and methods described herein enable minimally invasive surgical procedures in pedicle screw placement without the need for a trocar needle and/or guidewires.

The systems described herein are made of biocompatible material for surgical implantation such as stainless steel, titanium and titanium-based alloys, combination metallic alloys and the like; various plastics, ceramics, biologically absorbable materials, and the like. Other biocompatible materials that can be used to produce the systems of the invention are well-known to the skilled artisan.

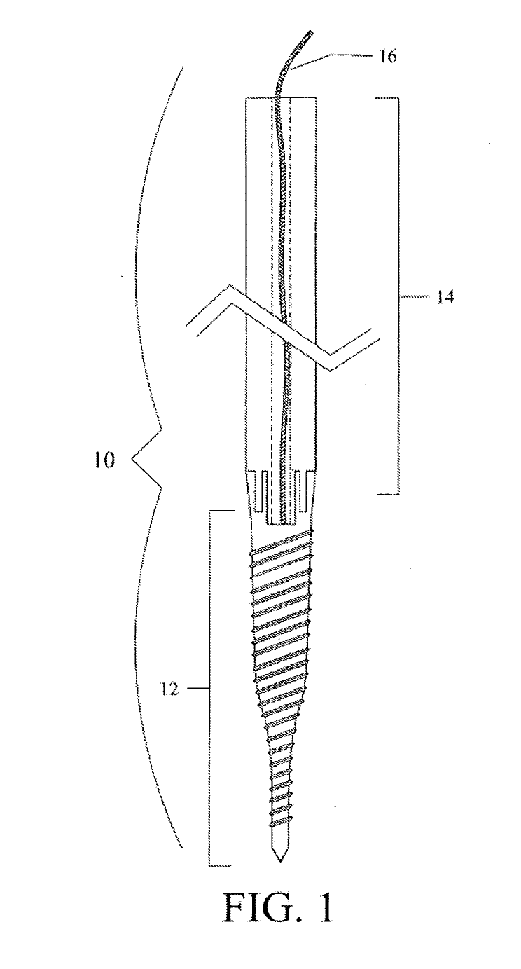

Referring to FIG. 1, a pedicle finder 10 is illustrated. The pedicle finder 10 comprises a pedicle anchor 12, an extender 14, and a flexible tether 16 that is attached to the pedicle anchor 12. Essentially, the pedicle finder 10 is a device that can have its tip member (pedicle anchor 12) be selectively attached or removed to or from bone.

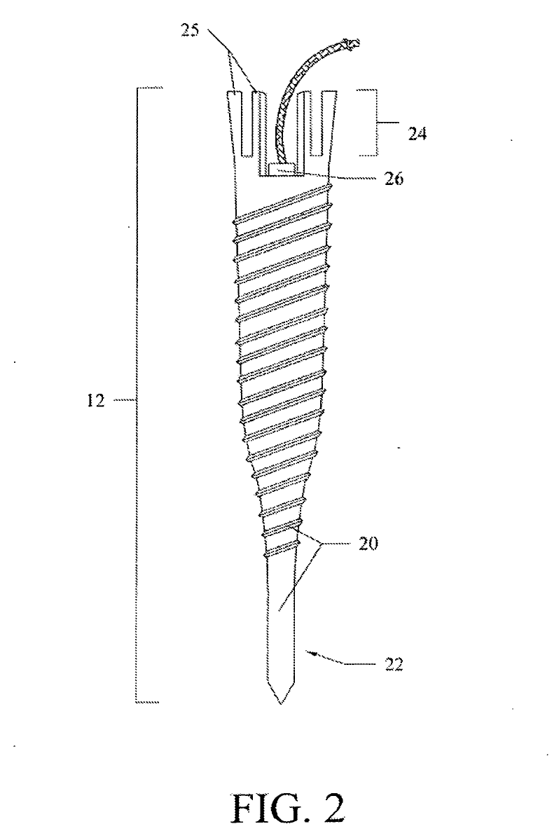

As shown in FIG. 2, the pedicle anchor 12 has a sharp tip 22 to engage and implant into bone. Along the external surface of the pedicle anchor 12 are threads 20 for boring into bone tissue. A spiral cutting thread can be used, but also other non-spiral cutting surfaces, such as a ribbed cone with tapered radiating relatively angled members for an even bore, or a conical rasp: In one embodiment, the sharp tip 22 includes threads 20 along a portion or its entirety. In other embodiments, the sharp tip 22 is free of threads so as to serve as an awl to penetrate cortical bone before engagement of the threads 20.

A top end 24 of the pedicle anchor 12 has at least one finger 25 to form a square jaw clutch arrangement. The finger(s) 25 of the pedicle anchor 12 interlocks with corresponding finger(s) 35 of a square jaw clutch arrangement of an extender 14 when the extender 14 is to be attached to the pedicle anchor 12 to prevent relative rotational movement between them.

While the rotation locking mechanism is illustrated using fingers 25, 35, it is not limited to this example. It is understood that any engageable arrangement can be used as a rotational locking means. For example, in certain embodiments, rather than finger(s) 25, the pedicle anchor 12 has a key fitted to be inserted into a corresponding lock located in the extender 14. When the key is inserted into the lock, relative rotational movement between the extender 14 and pedicle anchor 12 is prevented. Additional examples include, but are not limited to, the pedicle anchor. 12 having a complementary hex socket receptacle to a hex extension of an extender 14.

In a preferred embodiment, the sharp tip 22 of the pedicle anchor has at the most a 2 mm wide diameter. The remaining body of the pedicle anchor 12 can have a 5 mm wide diameter.

A flexible tether 16 can be attached anywhere on the pedicle anchor 12, so long as it can remain securely attached to the pedicle anchor 12 during use. Preferably, the flexible tether is attached to base 26 located at the top 24 of the pedicle anchor 12. In certain embodiments, the flexible tether is a chain made of biocompatible metallic alloys. In alternate embodiments, the flexible member can be made of biocompatible elastomeric materials, woven fibers, drawn fibers, or a combination thereof. The flexible tether should be of sufficient length to traverse from the pedicle anchor and out of the body of the patient for ease of securement.

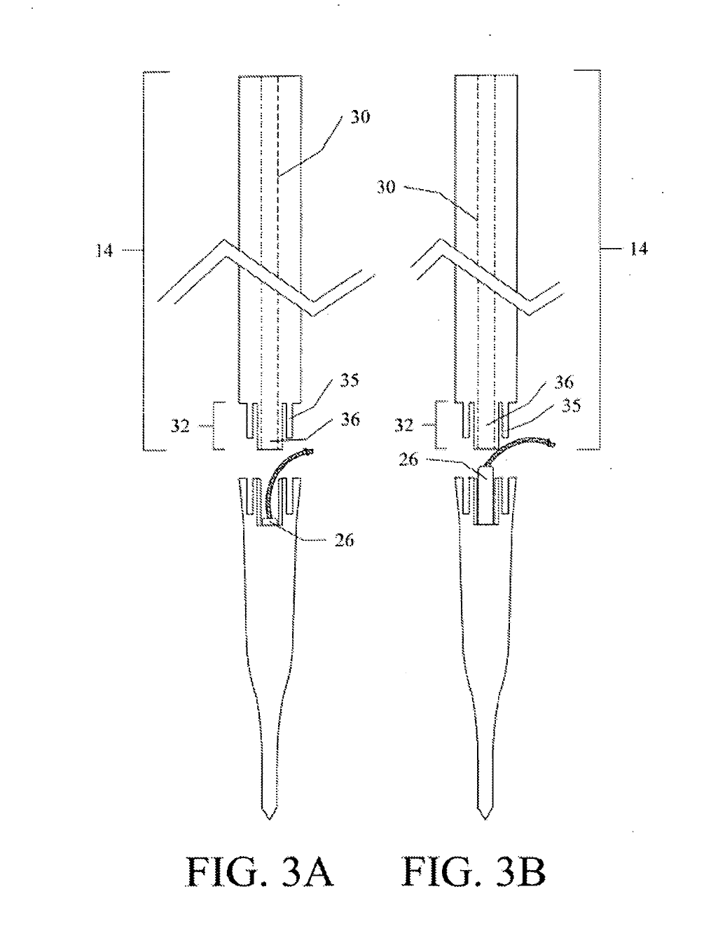

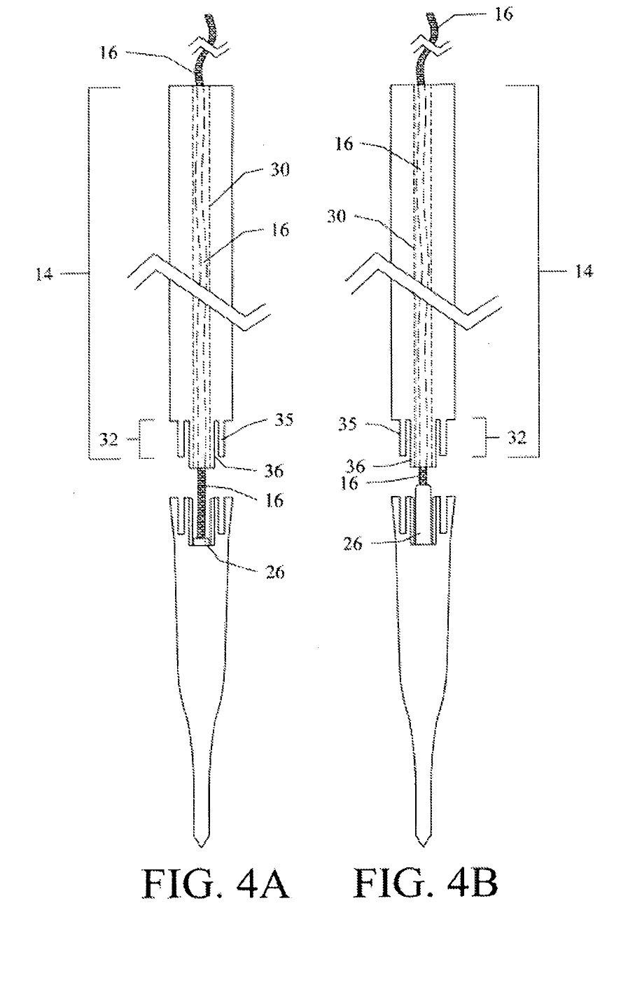

Referring to FIGS. 3A and 3B, an extender 14 that is detached from a pedicle anchor 12 is illustrated. The extender 14 comprises a main body seen as a cylindrical barrel shaped body with a circular or oval cross-section, but need not be. For example, the main body could have any envisioned cross-section shape, such as a triangle, square, rectangle, pentagon, hexagon, octagon, and the like. The main body has a terminal end 32 with at least one finger 35. Through the entire interior main body of the extender 14 is a hollow passage 30 through which a flexible tether may freely traverse (see FIGS. 4A and 4B). The extender further includes a tube 36 that is fitted over base 26 to aid in alignment of the extender 14 with the pedicle anchor 12. While the tube 36 and base 26 are optional and not required for attachment of the extender 14 to the pedicle anchor 12, both the tube 36 and base 26 when present will assist in concentric alignment of the extender 14 with the pedicle anchor 12 such that the only other alignment is rotational to align and lock the fingers 25, 35.

In one embodiment, the extender 14 includes a slit along the entire length of the main body, wherein the slit provides access to the hollow passage 30 from the exterior main body of the extender. The slit enables the user to easily slide the flexible tether into the hollow passage 30 without having to thread the flexible tether through the hollow passage.

As can be seen with FIG. 1, when the pedicle anchor 12 is to be attached to extender 14, the fingers 25, 35 slide together and are engaged such that any rotational force applied to the extender 14 will be transmitted to the pedicle anchor 12.

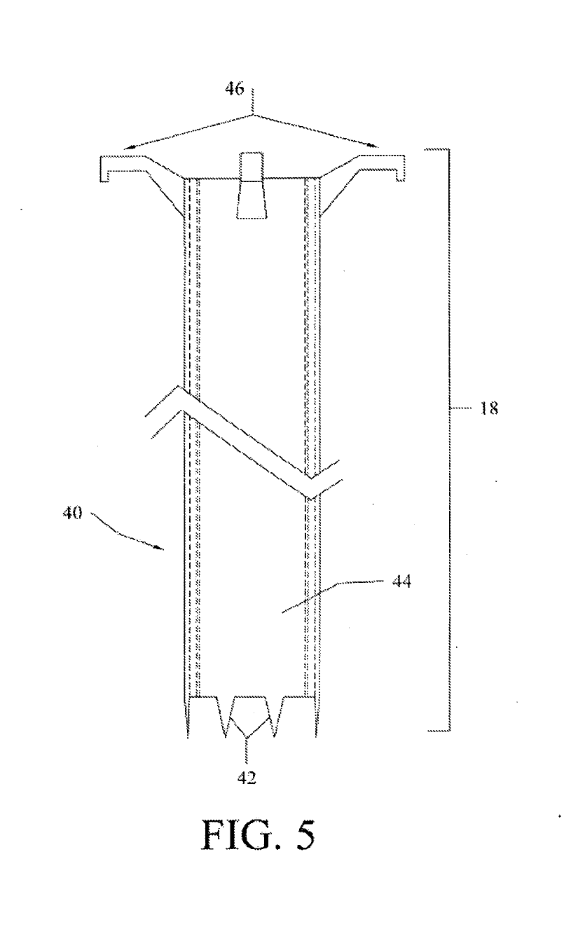

Referring to FIGS. 5. and 13, a dilator 18 is illustrated. The dilator 18 has a main body 40 that includes a hollow passage 44 through which an extender 14 may easily and freely traverse. At one end of the main body 40 are several sharp points 42 for insertion into bone 5. At the opposite end, the dilator 18 further includes at least one arm attachment 46.

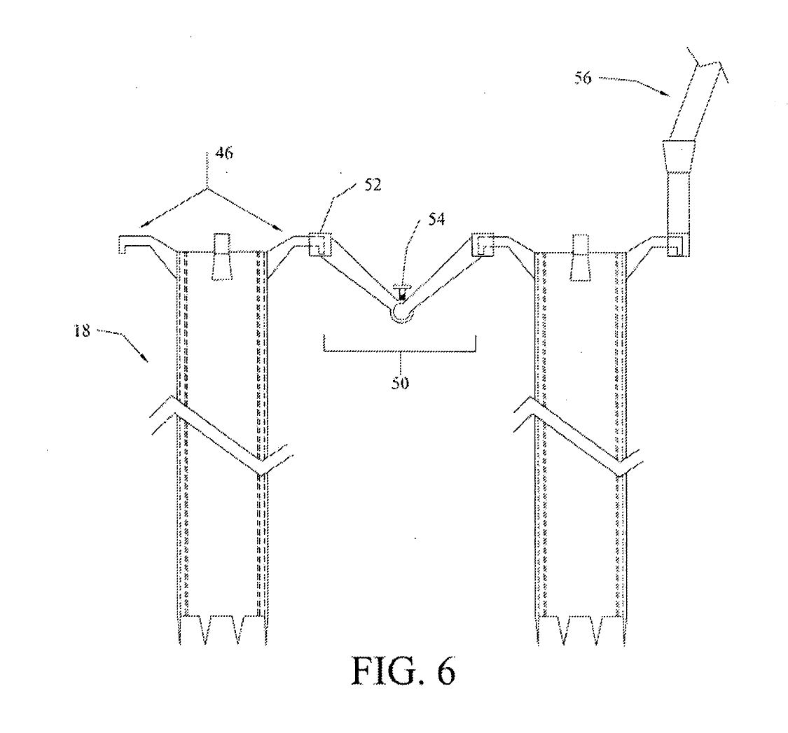

As illustrated in FIG. 6, dilators 18 can be interconnected to each other. To interconnect dilators 18, a linking element 50 is provided, where the linking element 50 has two ends 52 that attach to dilator arm attachments 46 and a lockable ball joint 54. Alternatively or in addition to the linking elements 50, the dilators 18 can be secured to a non-moving object via a fixation element 56. The fixation element 56 can be an articulated (as illustrated) or non-articulated universal arm adapted to attach to a dilator arm attachment and to a non-moving object such as an operating table so as to fixate the position of the dilator. The fixation element 56 is used to hold the dilators 18 in a fixed position. In certain instances, dilator positions need to be maintained for additional work to be performed on the body (i.e., interbody fusion, decompression, etc.). Alternatively, dilator positions are set via fixation element 56 or linking elements 50 so as to maintain the location of the pedicle in which a pedicle screw is to be inserted.

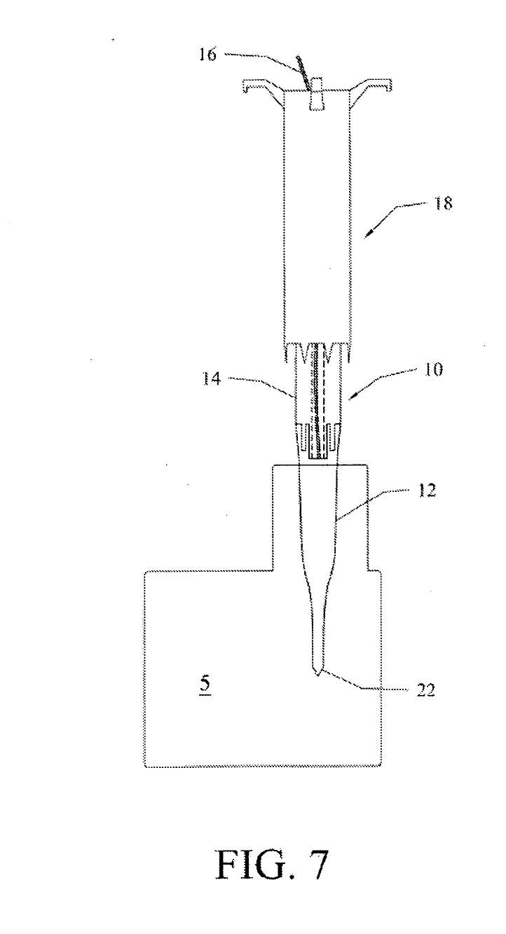

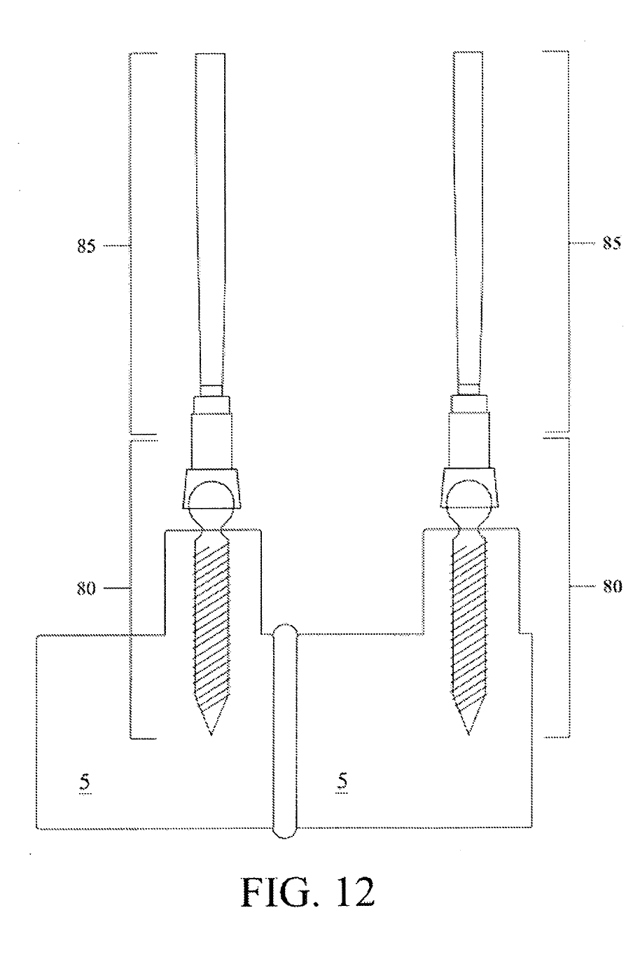

In a method of use, as illustrated in FIGS. 7-12, the pedicle finder 10 is placed into the pedicle either percutaneously or using a mini-open technique by using fluoroscopy. The sharp end. 22 of the pedicle anchor 12 penetrates the cortical bone and is then screwed into the pedicle. As the pedicle anchor 12 is screwed into the pedicle, the shaft widens and “taps” the pedicle, securely imbedding the pedicle anchor 12 in the pedicle and .also facilitating later placement of the pedicle screw.

Once the pedicle finder 10 is securely placed into the pedicle (e.g., 35 mm of the threads are in the pedicle), the extender 14 can be detached and slid off of pedicle anchor 12. The flexible tether 16 is then secured out of the way so that the decompression or interbody work can be performed at this time. In this manner, the position of the pedicle is safely maintained while any other work is performed.

Once the other work is complete and it is time to place the pedicle screw into bone, the flexible tether 16 attached to the pedicle anchor 12 is used to re-attach the extender 14 to the pedicle anchor 12. The flexible tether 16 simply slides through the hollow passage 30 of the extender 14 and the rotational locking mechanism is engaged between the extender 14 and the pedicle anchor 12.

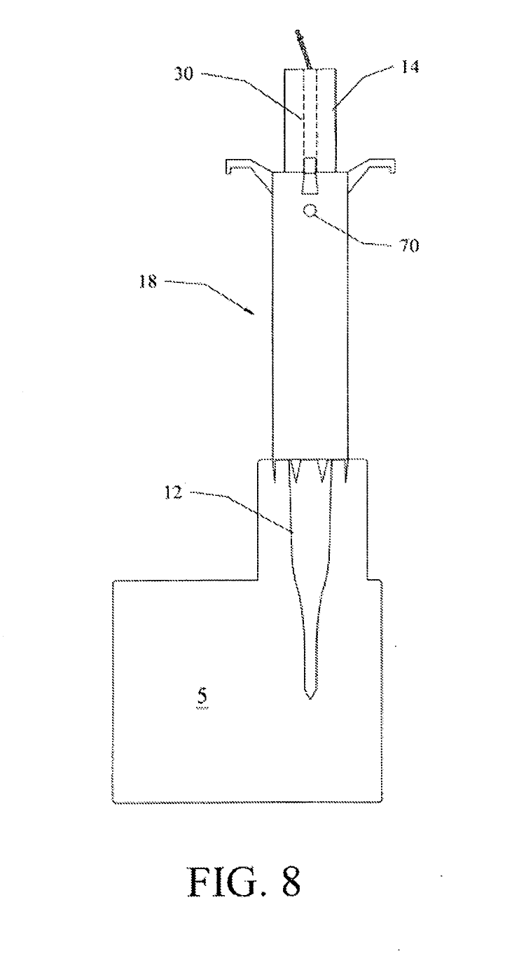

Once the extender 14 is re-attached to the pedicle anchor 12; the dilator 18 is slid over the pedicle finder 10, see FIG. 7. The teeth 42 at the end of the. dilator 18 serve to secure the distal end of the dilator into the bony lateral facet/transverse process of the vertebra to prevent the dilator from dislodging. As illustrated in FIG. 8, once the dilator 18 is in place, a screw 70 is used to lock the dilator 18 to the pedicle finder 10 so that the two are secured together.

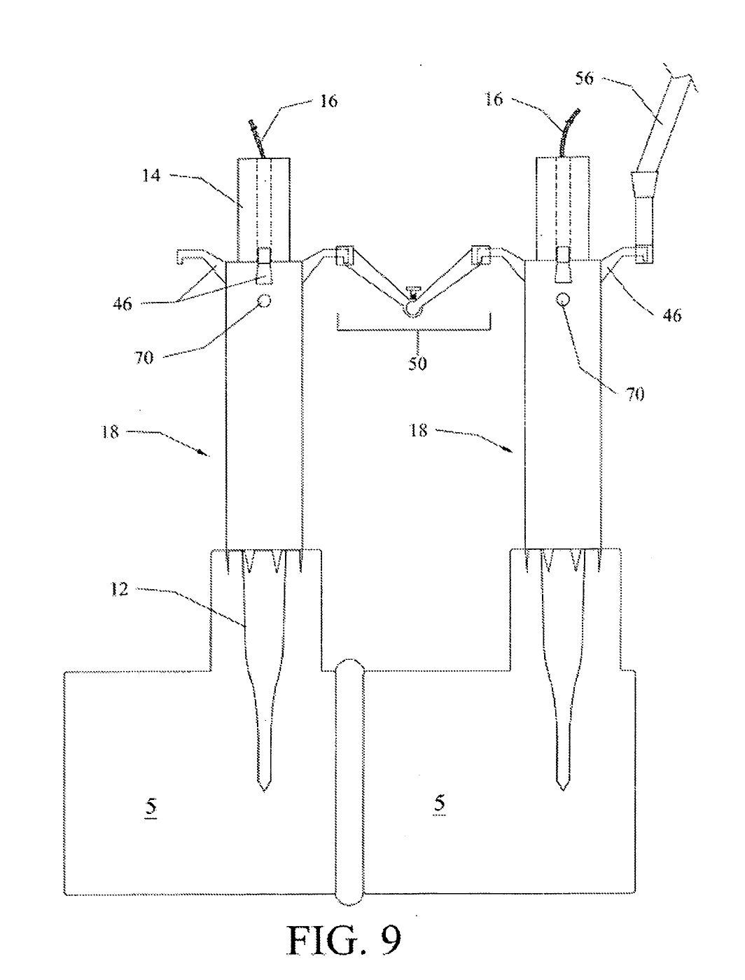

At the adjacent vertebral level, a pedicle finder and dilator are placed and secured together, see FIG. 9. In one embodiment, a linking element 50 (e.g., 5 cm in length) having a lockable ball joint 54 is then. attached to an arm attachment 46 on each of the dilators 18. The dilators 18 can be further secured, such as to a table, via a fixation element 56. Both the linking element 50 and fixation element 56 are then locked, securing all the dilators and pedicle-finders in place.

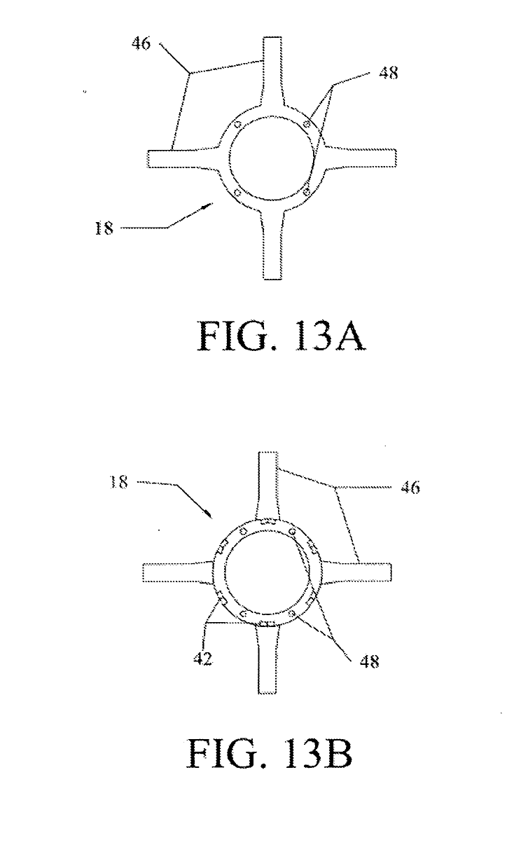

In certain embodiments, a narrow-diameter (1 mm) threaded securing wire can also be passed down a hollow passage 48 in the wall of the dilator tube (as seen best in FIGS. 13A and 13B) into the lateral facet joint to a depth of a few millimeters as an extra means of holding the dilators in place. Each dilator tube would accommodate-at least one wire.

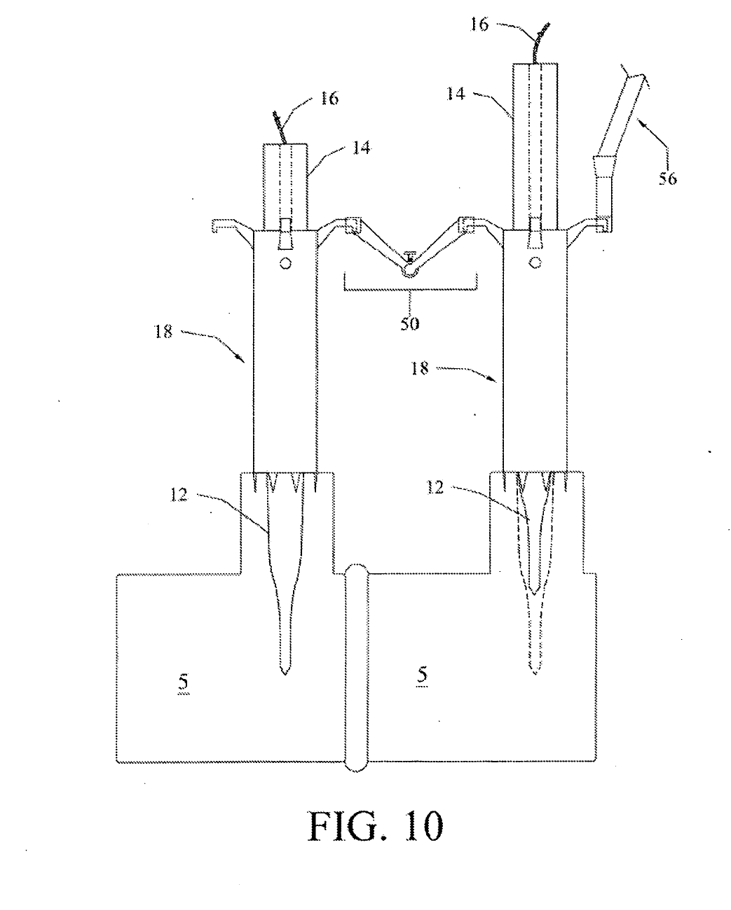

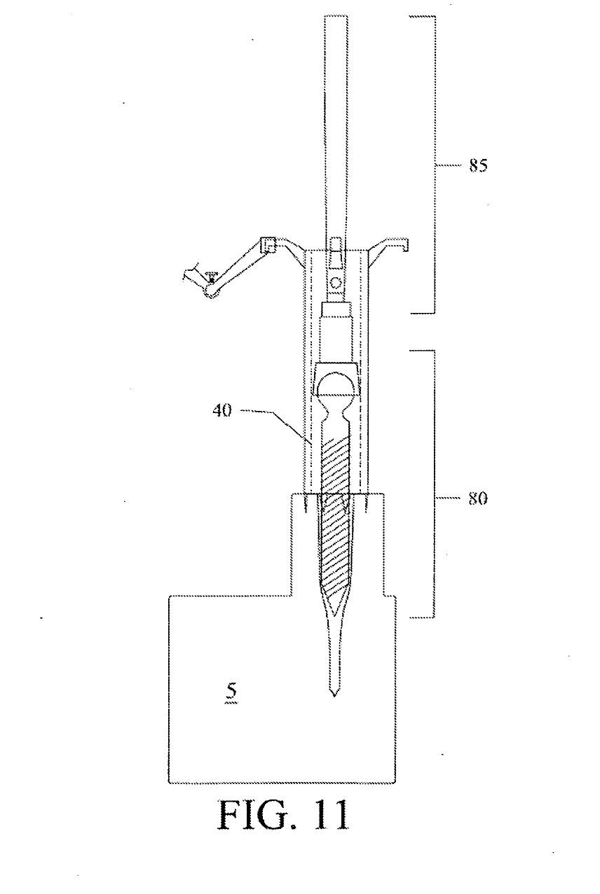

Once the dilators 18 have been secured, the pedicle finder 10 is unlocked from the dilator 18 and then the pedicle anchor 12 is unscrewed from the pedicle, see FIG. 10. The dilator 18 remains in place, secured by the multiple mechanisms described (such as linking element 50 and fixation element 56). A pedicle screw 80 is then slid down the hollow passage 40 of the dilator via a pedicle screw extender 85 and screwed into the pedicle via the tap hole created by the pedicle anchor 12, see FIG. 11.

The dilators, along with the securing wires and connecting arms, are all removed, leaving the pedicle screws along with their extenders in place, see FIG. 11.

All patents, patent applications, provisional applications, and publications referred to or cited herein, supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

I claim:

1. A pedicle screw placement system comprising a pedicle finder, wherein the pedicle finder comprises a pedicle anchor, a flexible tether attached to the pedicle anchor, and an extender removably attached to the pedicle anchor, wherein the pedicle anchor comprises a sharp conical portion and an extender attachment portion, wherein the extender comprises a pedicle anchor attachment portion and a passage to accommodate the tether, and wherein the extender attachment portion removably engages the pedicle anchor attachment portion to provide a rotation locking mechanism.

2. The pedicle screw placement system of claim 1, wherein the sharp conical portion of the pedicle anchor further comprises threads for boring into bone tissue.

3. The pedicle screw placement system of claim 1, wherein the flexible tether is made from any one or more materials selected from the group consisting of: biocompatible metallic alloys, elastomeric materials, woven fibers, and drawn fibers.

4. The pedicle screw placement system of claim 1, wherein the extender further comprises a slit with access to the passage that accommodates the tether.

5. The pedicle screw placement system of claim 1, further comprising a dilator, wherein the dilator includes a passage to accommodate the extender and a first end that includes a means for securing the dilator into bone.

6. The pedicle screw placement system of claim 5, wherein the dilator includes a second end comprising at least two attachment arms.

7. The pedicle screw placement system of claim 6, further comprising a linking element attached to at least one attachment arm.

8. The pedicle screw placement system of claim 7, wherein the linking element is a lockable ball joint.

9. The pedicle screw placement system of claim 5, wherein the dilator includes a second end comprising a fixation element.

10. The pedicle screw placement system of claim 5, further comprising a locking mechanism to lock the dilator to the pedicle finder.

11. The pedicle screw placement system of claim 5, wherein the dilator further comprises a passage to accommodate a securing wire.

12. A method for placing a. pedicle screw into bone comprising the steps of:
(a) providing a plurality of pedicle screw placement systems, wherein the pedicle screw placement system comprises: a dilator and a pedicle finder, wherein the pedicle finder comprises a pedicle anchor, a flexible tether attached to the pedicle anchor, and an extender removably attached to the pedicle anchor, wherein the pedicle anchor comprises a sharp conical portion and an extender attachment portion, wherein the extender comprises a pedicle anchor attachment portion and a passage to accommodate the tether, wherein the extender attachment portion removably engages the pedicle anchor attachment portion to provide a rotation locking mechanism, and wherein the dilator includes a passage to accommodate the extender and a first end that includes a means for securing the dilator into bone;
(b) penetrating the sharp portion of the pedicle anchor of a pedicle finder into a pedicle and forming a tap hole;
(c) detaching and/or attaching the extender to/from the pedicle anchor using the tether;
(d) sliding the dilator over the pedicle finder and securing the dilator to the pedicle with the first end;
(e) removing the pedicle finder from the dilator; and
(f) placing and screwing a pedicle screw into the tap hole.

13. The method of claim 12, wherein the sharp conical portion of the pedicle anchor further comprises threads that assist in penetrating the sharp portion into the pedicle.

14. The method of claim 12, wherein the flexible tether is made from any one or more materials selected from the group consisting of: biocompatible metallic alloys, elastomeric materials, woven fibers, and drawn fibers.

15. The method of claim 12, wherein the extender further comprises a slit with access to the passage that accommodates the tether.

16. The method of claim 12, wherein the dilator includes a second end comprising at least two attachment arms and the pedicle screw placement system further comprises a dilator linking element, and the method further comprises a step following step (d) and prior to step (e) of linking the attachment arms of two or more dilators together using dilator linking elements.

17. The method of claim 12, wherein the dilator includes a second end comprising a fixation element; and the method further comprises a step following step (d) and prior to step (e) of fixating at least one dilator to a non-movable object using the fixation element.

18. The method of claim 12, wherein the pedicle screw placement system further comprises a locking mechanism to lock the dilator to the pedicle finder.

19. The method of. claim 12, wherein the dilator further comprises a passage to accommodate a securing wire, and the method further comprises a step following step (d) and prior to step (e) of passing the securing wire down the accommodating passage of the dilator and inserting the securing wire into the pedicle.
